(19) United States
(12) Patent Application Publication
(10) Pub. No.: US 2010/0021915 A1
(43) Pub. Date: Jan. 28, 2010

(54) HIGH THROUGHPUT DNA SEQUENCING METHOD AND APPARATUS

(75) Inventors: Rishi Lee Khan, Philadelphia, PA (US); James Stephen Schwaber, Wilmington, DE (US)

Correspondence Address: DAVID S. RESNICK, NIXON PEABODY LLP, 100 SUMMER STREET, BOSTON, MA, 02110-2131, US

(73) Assignees: THOMAS JEFFERSON UNIVERSITY, Philadelphia, PA (US); UD TECHNOLOGY CORPORATION, Newark, DE (US)

(21) Appl. No.: 12/518,332

(22) PCT Filed: Dec. 11, 2007
(86) PCT No.: PCT/US2007/025242

§ 371 Date: Jun. 9, 2009

Related U.S. Application Data
(60) Provisional application No. 60/873,943, filed on Dec. 11, 2006.

(51) Int. Cl. C12Q 1/68; C12M 1/34

(52) U.S. Cl. 435/6; 435/287.2

(57) ABSTRACT

The present invention relates to a method for high throughput nucleic acid sequencing using a multi-bead flow cell and pyrophosphate sequencing, a sequencer capable of performing this method, and a kit of the pyrophosphate sequencing reagents.

### CROSS REFERENCE TO RELATED APPLICATION

This application claims benefit of U.S. provisional application No. 60/873,943, entitled “Digital Platform for Gene Expression Measurement,” filed Dec. 11, 2006, the contents of which are incorporated herein by reference in the entirety.

### FIELD OF THE INVENTION

The present invention relates to high throughput DNA sequencing using a multi-bead flow cell and pyrophosphate sequencing.

### BACKGROUND OF THE INVENTION

High throughput methods, such as transcript microarrays, offer the ability to gain insight into the function of biological systems through concurrent measurement of system-wide responses to various stimuli. They also have the potential to identify genes, or functionally associated clusters of genes, that can serve as diagnostic biomarkers. Through these kinds of systems biology and biomarker studies, microarray methods could be highly useful in the approach to understanding, treating, or managing the effects of many diseases.

In principle, microarray-based genomic analysis allows the study of system-wide effects of stimuli or disease, candidates for disease biomarkers, and discovery of novel genes. Currently microarray-generated data has been shown to be inconsistent between different laboratories, however, perhaps due to the analog and relative nature of gene expression measurement and the variability introduced by the various steps in the process. For example, microarray experiments contain many steps that may introduce variability including RNA amplification, labeling, hybridization, and slide printing. Also, microarrays require probes (clones or oligonucleotides) and detect only the expression of genes corresponding to the probes. Further, different laboratories use different probes on microarrays, making comparisons between laboratories difficult.

Amplification of small amounts of cellular RNA, the upfront costs of probe manufacture and storage, and the need for independent confirmation of microarray-based results limit the potential for global gene expression datasets. These limitations hinder the development of systems-biology level models of integrated gene functions as well as identification of reliable biomarkers.

Hence, there remains a need for more precise, more reliable, and less expensive high throughput approach to gathering data on genomic expression

### SUMMARY OF THE INVENTION

The present invention provides for a novel process for high throughput sequencing of nucleic acid molecules, such as DNA.

The present invention provides for a novel device for high throughput sequencing of nucleic acid molecules, such as DNA.

The present invention provides novel kits for high throughput nucleic acid sequencing, such as DNA sequencing.

These and other aspects of the present invention have been achieved by the discovery by the present inventors of a one-well, multi-bead sequencing system.

### BRIEF DESCRIPTION OF THE DRAWINGS

FIG. 1: Schematic over of an example of a sequencer according to an embodiment of the present invention.

FIG. 2: Schematic of how the DNA sequences are applied to beads in an embodiment of the present invention.

FIG. 3: Distribution of number of pyrosequencing flows (e.g., the introduction of one nucleotide) before detecting a base for a homogenous population (top) and a population of two distinct sequences on a bead (bottom). Using these distributions, beads with non-homogenous populations are identified.

FIG. 4: Schematic of a multi-flow cell example embodiment of the present invention.

FIG. 5: Depicts the percent unique sequences (beginning with CATG) in non-redundant human RefSeq with given sequence lengths. Seventeen bases uniquely identify 95% of the genes. Thirty-five bases uniquely identify 99% of the genes. Fifty bases uniquely identify 99.8% of the genes.

### DETAILED DESCRIPTION OF THE INVENTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.” The term “about” when used in connection with percentages may mean±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

One application of the present embodiments provides for measuring gene expression by sequencing small portions of nucleic acid and mapping them to pre-existing genomic knowledge. More specifically, the process isolates individual mRNA molecules, amplifies each of these on a solid substrate, and determines the sequence of the amplified template in a high throughput manner. This approach comprises global molecular biology methods, digital microscopy, image analysis, bioinformatics and computational analyses. The approach presented herein may prove less expensive than existing technologies while providing accurate, reproducible, and higher throughput.

Current DNA sequencing technologies such as the Genome Sequencer FLX (454 Life Sciences/Roche Applied Science), the SOLiD™ system (Applied Biosystems/Applera Corp.), and the Illumina Genome Analyzer, powered by Solexa® (Illumina, Inc.) are all rather slow (e.g., 12-15 Megabases/hour) and expensive (e.g., $7-$70/Megabase) to be applied widely to whole genome sequencing or other applications such as assays for gene expression, miRNA expression, promoter binding, or genome-wide searches for single nucleotide polymorphisms.

One approach to current sequencing techniques is to create instruments that take advantage of pyrosequencing. The 454 Life Sciences' technology allows for long reads enabling de novo whole genome sequencing while other technologies provide higher throughput and lower cost. Unfortunately, 454 Life Sciences' technology requires a significant number of photons to reach the CCD, and thus uses fiber optics to guide the light to the CCD. This eliminates this methodology's ability to cheaply use multiple flow cells. Diffusion of the pyrophosphate during pyrophosphate sequencing reactions provides too much cross-talk between beads, and thus current technologies require microwells to physically separate the beads. The 454 Life Sciences' solution to this problem is their PicoTiter™ plate comprising millions of etched fiber optic channels epoxied directly to a CCD. This limits their read count, however, (max 1.6 million-currently 0.4 million) and the larger reaction volumes (>200 μl per nucleotide flow) increase cost. Other competitors avoid pyrosequencing or other methods of endogenous sequencing by synthesis, because these methods require real time image capture and thus large image acquisition by multiple sweeps with microscope-based cameras are not possible.

Another approach, taken by Applied Biosciences, sequences by ligation on beads covalently attached to a glass substrate (a slide). A fluorescently labeled oligonucleotide (8-mer) with specific bases in positions 4 and 5 are incorporated into the existing sequence on the beads. The oligonucleotide is capped on the 3′ OH end such that no further ligation can be performed. The oligonucleotide is cleaved and the process is repeated five or more times. This system can read a massive 240 million beads with an average length of 25 bases (possibly extendable to 35 bases). The major advantages of this machine are the megabases-per-hour throughput and the cost per base. The major disadvantage is short read length (i.e. typically 25 bases and always less than 50 bases per sequence), which makes de novo sequencing problematic (even with mate-paired reads). Although this method may be useful for whole genome resequencing, the recent Craig Venter and James Watson genomes suggest that large indels and transpositions are common among individuals and these will be hard to detect with 25 base-35 base reads. Another major disadvantage of this system is the large amount of raw data produced by the system and, consequently, the amount of post processing necessary to produce sequence data.

A third DNA sequencing approach is Illumina's Solexa® system. This system immobilize primers on a slide and grows clusters of homogeneous sequence template. The clusters are sequenced by synthesis of fluorescently labeled nucleotides. The Solexa® system has many of the same advantages and disadvantages of Applied Biosciences' SOLiD system, but provides 16% of the throughput for a similar cost.

The present invention differs from other approaches by providing higher quality (e.g., length) and quantity reads by sequencing by synthesis using dNTP's to produce long reads (i.e. >250 bp) while using flow cells and immobilization technologies to maximize the number of parallel reads (e.g, ˜40 million). The present invention can use off-the-shelf optics and microfluidics to minimize capital (e.g., <$100,000) and operational (e.g., <$1000/run) costs of sequencing. Beyond whole genome sequencing, the present invention provides an alternative to qualitative and noisy microarray, promoter binding, and miRNA high throughput assays with digital readouts of sequences. In certain circumstances, it may even be cost effective to replace quantitative real-time polymerase chain reaction (PCR) assays.

The embodiments of the present invention have been designed to avoid the pyrophosphate diffusion issue by capturing images before pyrophosphate can diffuse too far from the beads (e.g., 1/10th of a second) and deconvolving the obtained images to determine the beads that produced the light. The pyrosequencing reaction as used in the present invention can be optimized to produce a large amount of light (e.g., by removing apyrase) in a very short time frame (e.g., by optimizing the effective concentrations of a pyrophosphate to ATP converting enzyme such as ATP sulfurylase or thermostable ATP sulfurylase, and an ATP to detectable signal converting enzyme such as luciferase). The present invention can image the sequencing reactions with off the shelf SLR cameras using macro lenses and using commercially available beads (e.g., 4.5 μm beads). The end result is that the present invention is expected to have the capacity of reading 1.6 million sequences of 250 bases in parallel per flow cell. With the inclusion of more than one flow cell per sequencer, washing overhead time and machine cost can be reduced.

In an aspect of the present invention, DNA is sequenced by the following steps: (1) clonal populations of a DNA sequence are created on millions of beads; (2) populations of millions of beads are immobilized to glass in a microfluidics chamber; (3) a single nucleotide and various reagents flow over the beads; (4) complement base incorporation at individual beads is signaled by chemiluminesence; (5) the signal is captured by an image acquisition system (e.g., a camera); (6) beads are washed; (7) steps 3-5 are repeated for all desired nucleotides as many as hundreds of times; and, (8) the sequence on each bead is determined by image analysis in parallel over millions of beads.

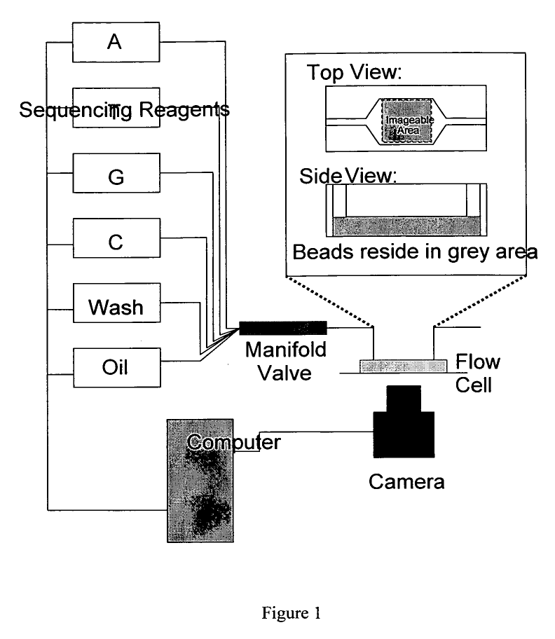

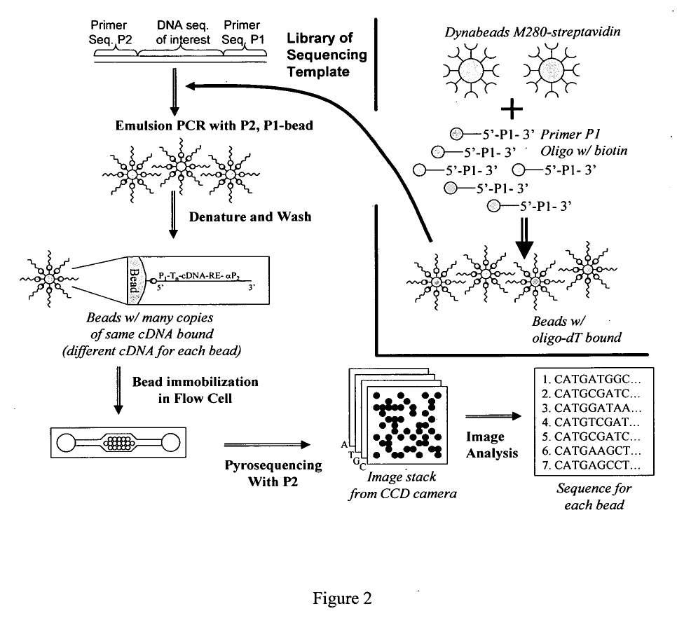

The present invention provides for a novel process and device for sequencing nucleic acids, such as DNA. Essentially, the present invention provides a one-well, multi-bead sequencer. An overview of one example of the present invention is shown in FIG. 1 and FIG. 2. The present invention uses standard molecular biology techniques to convert RNA or DNA molecules into a double stranded DNA molecule flanked by known sequences on both ends (primer sequences P1 and P2) as shown at the top of FIG. 2. Each cDNA molecule can be isolated to an individual microenvironment and amplified by PCR to create homogeneous sequencing templates through a method called emulsion PCR. This microenvironment can be achieved by creating a water-in-oil emulsion with the aqueous phase containing all of the necessary PCR reagents and template. Magnetic beads covered, for example, with hundreds of thousands of copies of one of the PCR primers (primer P1) can be used as a solid substrate for the PCR reaction. This method is called BEAMing (bead, emulsion, amplification, and magnetics). Emulsion PCR for deriving clonal DNA populations on beads has been discussed broadly in the literature. After the emulsion PCR step, each bead can either be coated with nothing or coated with a homogenous population of DNA with sequence flanked by primer sequences P1 and P2. The emulsion can be broken and beads with sequences can be enriched by hybridizing with larger capture beads and centrifuged through a glycerol gradient. The beads can then be immobilized to the slide inside the flow cell. The flow cell can be placed in an imaging instrument and each nucleotide triphosphate flowed through the flow cell individually and separated by washing steps, if desired. Images can be taken as the pyrosequencing reaction occurs on the beads. After the reactions are complete, the time series of images are processed and the sequences on each bead are determined.

In another aspect, the present invention sequences the template on the beads using pyrosequencing on an imagable surface (e.g., a microscope coverslip) imaged by a complementary metal-oxide-semiconductor (CMOS) camera. When DNA polymerase incorporates a nucleotide into the complementary strand of the sequencing template, it releases a pyrophosphate (PPi). In pyrosequencing, this pyrophosphate is converted into ATP by ATP sulfurylase (or other PPi to ATP converting enzyme such as pyruvate orthophosphate dikinase), which produces the energy required for luciferase to oxidize luciferin and generate light.

The present invention provides a number of potential improvements over the current state of the art. First, apyrase is removed from the reaction to increase the rate and amount of signal produced by the reaction. Apyrase is used in current state of the art pyrosequencing because it stops the signal generation and allows the next base to be added. Because the beads are immobilized, they can simply be washed without loss of beads. Second, the concentrations of the reaction components have been modified greatly (see kit later contents for details) to increase the rate at which signal is produced. Third, a separate PPi to ATP converting enzyme, pyruvate orthophosphate dikinase, can be used in this invention to decrease background noise from unwanted side reactions and increase signal generation rates.

In one embodiment, the present invention provides a method for sequencing a nucleic acid molecule (or molecules) including:
(a) providing a sequencer comprising:
   (i) a reservoir; and
   (ii) a microfluidic flow cell, comprising a flow chamber including a planar imagable area and a plurality of beads immobilized onto the planar imagable area;
   wherein the plurality of reservoirs is fluidly connected to the flow cell; and a substantial portion of the beads further include a plurality of nucleic acid molecules attached thereto, wherein the nucleic acid molecules present on an individual bead are homogeneous;
(b) contacting the nucleic acid molecule(s) with pyrophosphate sequencing reagents including:
   nucleotide triphosphate(s);
   a polymerase;
   a pyrophosphate-to-ATP-converting enzyme; and
   an ATP detecting enzyme; and
(c) detecting the resulting optical signals, wherein each optical signal is indicative of a reaction of pyrophosphate sequencing reagents with a target nucleic acid molecule on a bead, thereby sequencing the nucleic acid.

In another aspect, the sequencer further comprises a plurality of reservoirs (e.g., 2, 3, 4, 5, 6, 7, 8, 9, 10, or more). These reservoirs can contain the pyrophosphate sequencing reagents (e.g., the four individual nucleotide triphosphates and the enzymes), a wash solution (e.g., wash buffer) for washing the beads between each nucleotide introduction, and a carrier suitable to separate the aqueous sequencing reagents (e.g., mineral oil).

In another aspect, the sequencer further includes a plurality of flow cells (e.g., 2, 5, 10, 15, 20, 25, 30, 25, 40, 45, 50, or more).

In another aspect, the sequencer further comprises a plurality of reservoirs (e.g., 2, 3, 4, 5, 6, 7, 8, 9, 10, or more) and a plurality of flow cells (e.g., 2, 3, 4, 5, 6, 7, 8, 9, 10, or more). The flow cells can be connected to the same or different reservoirs.

In another aspect, it may be desirable for the pyrophosphate sequencing reagents to contain other than apyrase.

The substantial portion of beads further comprising a plurality of nucleic acid molecules means that at least about 50% of the beads have nucleic acid molecules attached thereto. Additional examples of “substantial portion” include approximately 60%, 70%, 80%, 90%, 95%, and 100%.

Flow cells may be made of polydimethylsiloxane (PDMS) and can be bonded to an imagable area (e.g., glass) by covalent linking after the application of oxygen plasma. The PDMS and imagable area (e.g., glass) can also be held in contact mechanically (e.g., a gasket and pressure setup). It is noted that one could also use PDMS as the imagable area. The tight bond to the imagable area allows pressurized flow without the issue of the PDMS losing adherence to the imagable area. An advantage of using glass for the imagable area is that glass is optically tuned to minimize distortion and maximize the number of photons traveling through and arriving at the detector (e.g., camera). Typically, a single inlet and outlet connect the reaction chamber inside of the flow cell to the rest of the microfluidics system. Reagents can be pumped using, for example, syringe pumps or pressurized gas and can be multiplexed into the flow cell using a manifold valve, which multiplexes the reagents so that they can traverse the flow cell one at a time. The reagents flow in the reaction chamber between the PDMS and the glass. The beads are immobilized to the glass on the side inside the reaction chamber. The reaction is imaged through the bottom of the glass in the imagable area (see FIG. 1).

The imagable area will typically depend on the signal detection device selected. If, for example, a camera is used, then the imagable area is selected to fit the aspect ratio of the camera (e.g., 1:1, 3:2, 4:3, or 16:9). Examples of the size of the imagable area include as small as 0.1 mm on one side to 50 mm on one side. An area tested in an example embodiment is a 7 mm×4.6 mm area.

Homogeneous populations of sequencable DNA can be attached to the bead via emulsion PCR as described by Dressman et al., Transforming single DNA molecules into fluorescent magnetic particles for detection & enumeration of genetic variations, 100 P.N.A.S. 8817-22 (2003). Sequencable DNA is defined as single stranded DNA flanked by two universal, known primer sequences (herein antisense P1 for the 5′ sequence and sense P2 for the 3′ sequence) as shown in FIG. 2. For example, first, the streptavidin coated beads are bound with a 5′ biotinylated primer (sense P1) through streptavidin-biotin binding. Typically this will yield 1 million to 20 million primers attached to each bead. Next, a water-in-oil emulsion is created such that PCR reagents (with a molar excess of primer P2 but a very small amount of primer P1) are in the aqueous phase. Emulsions are made with a given specific number of compartments (within +/−20%). Molecules of sequencable DNA and beads are added to the aqueous phase such that the ratio of beads to emulsion compartments (ratio P) and the ratio of molecules to emulsion compartments (ratio Q) fits the application in question. PCR is performed such that each emulsion compartment acts as a separate reaction chamber. After the emulsion PCR, the beads in a compartment that also contained a DNA molecule will be coated with nucleic acids that are the antisense strand of the starting DNA molecule. Typically Q is set to a low ratio such as 1% to 10% such that most of the beads with sequence contain homogenous sequence. P can vary from 1% to 200% depending on the application. In this case, the DNA sequence contains a sequence of from 10, 20, 30, 40, 50,60, 70, 80, 90, 100, 200, 300, 400, 500, 600, 700, 800, 900, 1,000, 2,000, 3,000, 4,000, 5,000, 6,000, 7,000, 8,000, 9,000 to 10,000 bases.

If it is desirable to capture and sequence a maximal number of molecules from a sample and the counts of each molecule sequence are important (such as in a gene expression study from a single cell), then P is extremely high such that on average, more than one bead is contained in a compartment and very few compartments have no beads. In this case, the DNA sequences contain a random sequence of 10, 11, 12, 13, 14, 15, 16, 17, 18, 19, to 20 bases (length determined at start of experiment and is logarithmically proportional to the number of molecules placed in the emulsion PCR) between the start of the sequence and the primer P2 sequence. During sequencing, if the same random sequence tag (of known length) is found for two same sequences, then it is known that these beads are redundant and should be only counted once.

After the emulsion PCR is complete, the double strand is broken by incubation with NaOH, beads are isolated to the side of a tube with a magnet, and supernatant is removed. Beads that contain sequences can be enriched as follows. Beads from the emulsion PCR are hybridized with larger, less dense nonmagnetic ‘capture’ beads coated with P2 sequence. These capture beads will capture the beads that contain antisense P2 (i.e. the beads that were coated with a DNA sequence during emulsion PCR). Beads bound with the capture beads will be less dense than unbound beads and can be separated by centrifugation through glycerol and keeping the supernatant (the unbound beads will form a pellet at the base of the tube). Enriched beads will typically contain 1 million to 20 million copies of a nucleic acid molecule per bead.

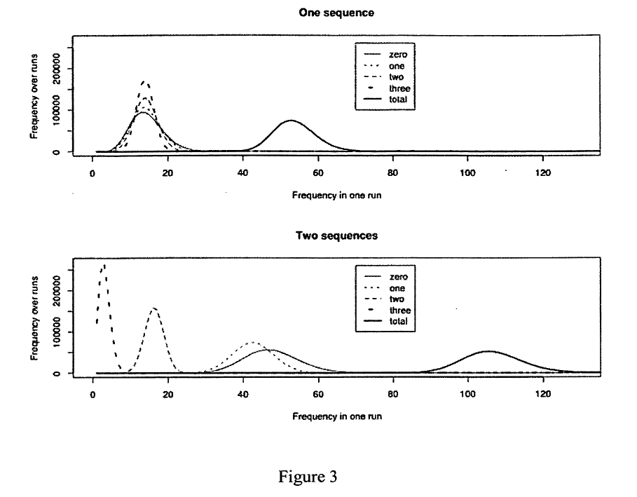

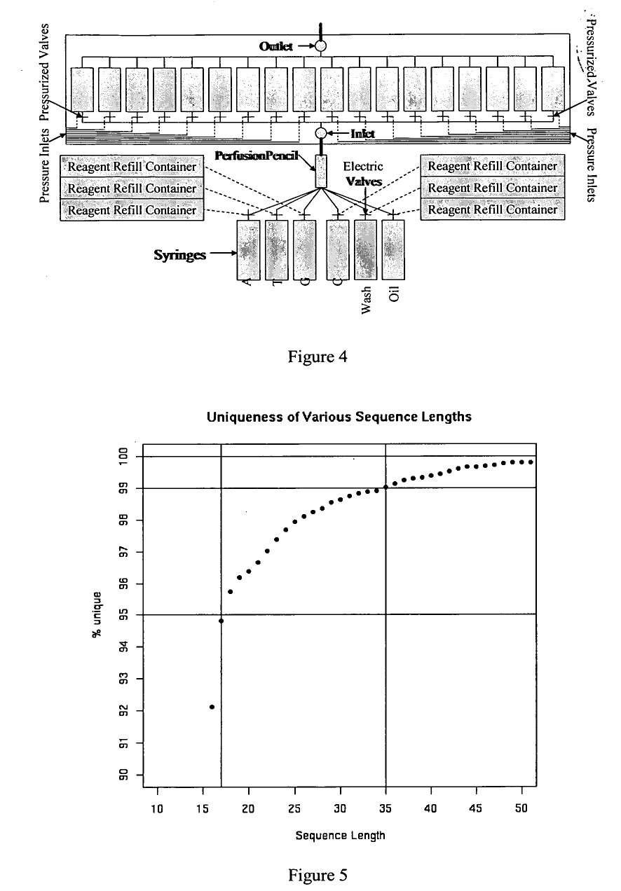

Homogeneous templates on a bead are desirable for sequencing because non-homogenous sequences yield spurious signals (e.g., each different sequence will produce signal as the next nucleotide in the sequence is flowed in the pyrophosphate sequencing reaction). Although the signals read from non-homogenous beads will be unusable, they can be flagged and removed from the end results by noting the distribution of nucleotide cycles before the next incorporation (see FIG. 3). In general, a bead with two sequences will yield data sequence twice as long as normal and the number of nucleotide flows between incorporation skews towards fewer flows.

In the present invention, when the signal detection device is a camera, then it usually is either a charged-coupled device camera (CCD) or a CMOS camera. The former is usually more expensive and tuned to low-light or high-frequency imaging, while the latter is usually consumer-grade and not as tuned to low light or high frequency imaging. In the present invention, the pyrophosphate sequencing reactions can be optimized such that the amount of signal (light) given off is sufficient for imaging with the less expensive CMOS cameras.

In another aspect, the microfluidic flow cell further comprises a first fluid inlet fluidly connected to the flow chamber and fluidly connected to the plurality of reservoirs; and a first fluid outlet fluidly connected to the flow chamber.

In another aspect, the first fluid inlet and first fluid outlet are connected to the same surface of the flow cell and are separated by the imagable area.

In another aspect, the planar imagable area comprises glass.

In another aspect, the space between the imagable area and the wall of the flow cell is from 5 μm to 100 μm.

Pyrophosphate sequencing has been shown to be able to sequence from 1 base to over 300 bases. On average, one cycle of four nucleotide flows yields 2.5 bases of sequence. The 454 Life Sciences' GS FLX sequencer runs 100 cycles and obtains 250 bases per sequence on average. In theory, there is no limit to the amount of sequence that can be generated (e.g., up to 10,000 or more bases). In practice, the length of sequence has been limited by the dephasing of signal due to incomplete incorporation of nucleotide or misincorporation when no incorporation should take place. Algorithms using Markov models have been created that model the dephasing and identify the correct sequence for small amounts of dephasing. See Eltoulhy et al., Modeling & Base Calling for DNA Synthesis, Proc. Int'l Conf. Acoustics, Speech, & Signal Processing (2006).

In another aspect, the contacting is performed by delivering the pyrophosphate sequencing reagents from the plurality of reservoirs to the flow chamber whereby the nucleic acids are exposed to the reagents.

In another aspect, the contacting further includes sequential delivery of homogeneous nucleotide triphosphates.

In another aspect, the pyrophosphate sequencing byproduct is detected by contacting it with an ATP sulfurylase under conditions that allow for formation of ATP. In another aspect, the ATP sulfurylase is a thermostable ATP sulfurylase.

In another aspect, the pyrophosphate sequencing byproduct is detected by contacting it with a pyruvate orthophosphate dikinase under conditions that allow for formation of ATP. In another aspect, the pyruvate is a thermostable pyruvate orthophosphate dikinase.

In another aspect, the method further comprises washing the flow cell with a wash buffer between each delivery of a nucleotide triphosphate. It may be desirable for the wash to further comprise apyrase. If apyrase is used, then the method can further includes a second washing of the flow cell with a wash capable of removing apyrase.

In another aspect, the nucleic acid primers are attached to the beads via their 5′ ends via a biotin-streptavidin binding linkage.

In another aspect, the beads are immobilized onto the imagable surface via a binding pair or a chemical bond, such that they do not move when reagents flow over them. If the beads move, it may be impossible to register the sequential images and determine the sequence of nucleic acid on each bead. Beads may be immobilized to the slide in a number of ways. One method of immobilizing beads is streptavidin-biotin binding of the beads to a biotinylated protein and covalent binding of carboxyl groups and amine groups of the protein to glass via silation of the glass with a reactive group containing silane (such as 3-aminopropyltriethoxysilane (APTES)). Another method is silanization of glass with APTES, but modification of the 3′ end of the DNA on the bead by ligation with a nucleotide containing a 3′ primary amine group and covalent bonding to the glass through amine-ester bonds.

In another aspect, the beads are immobilized to the imagable glass surface via a strepavadin-biotin-protein-silanyl linkage.

In another aspect, the beads are immobilized to the imagable glass surface via a 3′ nucleic acid comprising a primary amine group-silanyl linkage.

The dATP is an undesirable substrate for polymerase for nucleotide incorporation in pyrosequencing dATP, because it is also a substrate for the signal producing enzyme, luciferase, and thus creates a false signal. Instead, a thio-modified dATP can be used (deoxyadenosine-alpha-thiotriphosphate) that is a substrate for DNA polymerase but not luciferase. Additionally, thio-modified dCTP, dGTP, and dTTP can be used to decrease the misincorporation rate of the DNA polymerase. Ronaghi et al., Real-time DNA sequencing using detection of pyrophosphate release, 242 Anal. Biochem. 84-89 (1996). Further, the nucleotides can be capped on the 3′ side (e.g., with a 2-nitrobenzyl moiety) such that multiple nucleotides can not incorporate into a DNA strand if a homopolymer region exists. The 2-nitrobenzyl moiety can be removed through photolysis with 355 nm light. Wu et al., 3′-O-modified nucleotides as reversible terminators for pyrosequencing, 104(42) P.N.A.S. 16462-67 (2007).

The addition of nucleotide triphosphates can follow the standard four triphosphate rotation. If a desired sequence is sought, however, the addition of nucleotide triphosphates can be programmed or ordered to search for the desired sequence.

In another aspect, the diameter of the beads is from 1 μm to 20 μm. The diameter of the bead used in the present invention is limited only by the size of the flow cell, in particular, the space between the imagable area and the wall opposite the imagable area. Examples of bead diameter include 1, 2, 2.8, 3, 4, 4.5, 5, 6, 7, 8, 9, 10, 11, 12, 13, 14, 15, 16, 17, 18, 19, to 20 μm.

In another aspect, the beads are packed such that the free space is between the beads is less than 20% of the total imagable area.

One of the advantages of the present invention is the ability to densely pack the beads onto the imagable area. As opposed to well technology, which is limited to 1 bead per well, the present invention allows for a very large number of beads to be in contact with the imagable area. Maximal density of sphere packing on a planar surface (i.e., circular packing) is about 90.7%. Using the bead immobilization techniques described herein, approximately 80, 81, 82, 83, 84, 85, 86, 97, 88, 89, to 90% of the imagable area can be covered with a monolayer of beads.

In another aspect, the ATP detecting enzyme is luciferase, which produces light for detection. In another particular aspect, the luciferase is a thermostable firefly luciferase.

In another aspect, the signal detection is performed by a CMOS camera.

In another aspect, the optical signals from the pyrophosphate sequencing reaction are imaged before the reagents and byproducts diffuse far enough away from the bead incorporating the nucleotide sequence that the light can no longer be localized to that specific bead.

Pyrophosphate is generated at the surface of a bead during an incorporation event. However, there is a time delay before that pyrophosphate is converted into ATP (e.g., by ATP sulfurylase or pyruvate orthophosphate dikinase) and ATP is converted into a detectable signal (e.g., by luciferase). During this time, all substrates diffuse freely in the aqueous reaction media. As a result, some of the light generated from the incorporation event can be dispersed away from the bead surface. The amount of light generated away from the bead surface is non-linearly proportional to the amount of time the reaction has been occurring. Pyrophosphate diffuses at a rate of ˜700 μm²/sec and ATP diffuses at a rate of ˜300 μm²/sec. It is expected that the signal can be deconvolved and localized to a specific bead if the half-concentration iso-concentration contour (i.e., all positions where the concentration of signal is half of that at the bead surface) is less than 4 times the diameter of the bead. For example, if the beads are 4.5 μm in diameter, the signal must be captured before the pyrophosphate diffuses further than about 18 μm from the bead surface. In 100 Ms, the concentration of pyrophosphate about 15 μm from the bead surface is half of that at the bead surface. Therefore, the signal must be captured in 100 Ms.

One can adapt the model of the reaction-diffusion kinetics adapted from the literature (see, e.g., Agah et al., A multi-enzyme model for Pyrosequencing, 32(21) Nucleic Acids Res. e166 (2004)) and use it to simulate different concentrations of luciferase, ATP sulfurylase, and luciferin. Using this model, one can find the concentrations to generate enough signal in 100 Ms to be detected by the detection device (i.e., enough photons must reach the device such that the signal is at least twice the read noise). Examples of these concentrations are 3.3 mM for luciferin, 9 μM for ATP sulfurylase, and 3 mM luciferase. The reaction can generate 80,000 photons of light such that 640 photons reach the camera (f/#2.8, NA=0.18, 0.8% photons reach the sensor) over 223 pixels (15 μm diameter circle at 3.2× magnification covers 223 pixels of size 5.7 μm×5.7 μm). This produces an average of 2.9 photons per pixel in this region. At a quantum efficiency of 90% and internal gain of 5×, this yields 13 electrons/pixel, which is 3.0× the read noise floor (4.3 e-rms). These calculations change depending on the characteristics of the signal detector (e.g., camera) used.

In another aspect, the reaction is imaged within 10 Ms to 1000 Ms.

In another aspect, the optical signal is light and signal deconvolution is used to localize the light signal to a bead.

In another aspect, the sequencer comprises a plurality of flow cells. One sequencer can house multiple flow cells thereby increasing the number of megabases per run per machine. Further, the wash step of all of the flow cells can be combined, thereby reducing the run time. The wash step may take more than 80% of the run time (0.5 s for reagent delivery, 0.5 s for imaging (in 0.1 s intervals), and 5 s washing). A machine with a single flow cell can run and wash one base every 6 s whereas a machine with twenty-five flow cells can run and wash one base every 55 s (1 s for reagent delivery and reaction imaging per flow cell, plus 1 s for imagable area of next flow cell to enter the field of view of the camera per flow cell plus 5 s to wash all flow cells). This provides a>60% decrease in run time. For example, the machine may handle as many flow cells that can fit on the glass substrate. At the current time, a 48×60 mm commodity coverslip can hold forty-eight flow cells (4 mm across by 12 mm down). There is no limitation to the number of flow cells useable, however. In the limit, however, the run time per base is 2 s per flow cell and 18 flow cells achieves 90% of this limit and thirty-eight flow cells achieves 95% of this limit.

As noted above, detection of a signal can be performed within 100 Ms of the start of the reaction. Sometimes it may be difficult to synchronize the start of the reaction with the detection of the signal. Therefore, in an alternative aspect of the present invention, one may make multiple signal collections (e.g., pictures) in rapid succession and choose those with the most useable signal after the reaction has completed (either during run-time or offline on the computer during post-processing). The sequencing reaction will begin as soon as the reagents mix with the beads, and therefore, the beads closer to the flow cell inlet will begin reacting before the beads closer to the outlet. There are two ways to account for this occurrence: (1) minimize the effect by rapidly flowing the reagents over the beads in the flow cell (i.e., covering the flow cell in 1 Ms to 0 Ms) or (2) choose different signal collections (e.g., pictures) for different regions of the flow cell reaction chamber and use the data with the best signal for each region.

In another aspect, the present invention provides a novel kit comprising:
(a) a polymerase (e.g., at a concentration of 5, 6, 7, 8, 9, 10, 11, 12, 13, 14, 15, 16, 17, 18, 19, 20, 21, 22, 23, 24, 25, 26, 27, 28, 39, to 30 U/μL);
(b) a pyrophosphate-to-ATP converting enzyme (e.g., at a concentration of 5, 6, 7, 8, 9, 10, 11, 12, 13, 14, 15, 16, 17, 18, 19, 20, 21, 22, 23, 24, 25, 26, 27, 28, 39, to 30 mM, particularly when ATP sulfurylase is used);
(c) an ATP-detecting enzyme (e.g., at a concentration of 0.01, 0.05, 0.1, 0.5, 1, 2, 3, 4, to 5 mM);
(d) nucleotide triphosphates, or optionally nucleotide triphosphate analogues, optionally including, in place of dATP, a dATP analogue which is capable of acting as a substrate for a polymerase but incapable of acting as a substrate for a pyrophosphate-to-ATP converting enzyme;
(e) optionally, dideoxynucleotides, or optionally dideoxynucleotide analogues, optionally ddATP being replaced by a ddATP analogue, which is capable of acting as a substrate for a polymerase but incapable of acting as a substrate for a said PPi-detection enzyme;
(f) optionally, deoxynucleotides or dideoxynucleotides capped on the 3′ side with a 2-nitrobenzyl moiety to prevent successive incorporation of nucleotide in homopolymeric regions of DNA sequence.

In another aspect, the pyrophosphate to ATP converting enzyme is ATP sulfurylase or pyruvate orthophosphate dikinase. In another aspect, the ATP detecting enzyme is luciferase.

The amounts of pyrosequencing reagents useful in the present invention can be significantly different from those currently in use. For examples, the amount of polymerase, such as DNA polymerase, present can include a concentration of from 5, 6, 7, 8, 9, 10, 11, 12, 13, 14, 15, 16, 17, 18, 19, 20, 21, 22, 23, 24, 25, 26, 27, 28, 39, to 30 U/μL.

The amount of pyrophosphate to ATP converting enzyme can include from 5, 6, 7, 8, 9, 10, 11, 12, 13, 14, 15, 16, 17, 18, 19, 20, 21, 22, 23, 24, 25, 26, 27, 28, 39, to 30 mM, particularly when ATP sulfurylase is used.

The amount of ATP detecting enzyme can include from 0.01, 0.05, 0.1, 0.5, 1, 2, 3, 4, to 5 mM. The amount of nucleotide triphosphates can include from 0.1, 0.2, 0.3, 0.4, 0.5, 0.6, 0.7, 0.8, 0.9, to 1 mM. The examples of concentrations refer to the concentration of reagent to be delivered to a flow cell of the present invention.

In another aspect, the kit further includes (g) a pair of primers for PCR, at least one primer having means permitting immobilization of said primer; and (h) streptavidin-coated beads with primer pre-attached.

In another aspect, the kit further comprises (i) a pre-made flow cell including a PDMS structure bonded to a pre-silanated glass imagable area. Typically, a flow cell may be a one-time use object: after the beads are immobilized and sequenced, the flow cell can then be discarded.

One application of the present embodiments is useful to measure gene expression by sequencing small portions of nucleic acid and mapping them to pre-existing genomic knowledge. More specifically, the process isolates individual mRNA molecules, amplifies each of these on a solid substrate, and determines the sequence of the amplified template in a high throughput manner. This approach uses current molecular biology methods, digital microscopy, image analysis, bioinformatics, and computational analyses. The approach presented herein may prove less expensive than existing technologies while providing accurate, reproducible, and higher throughput.

The present invention takes advantage of standard molecular cloning techniques to convert mRNA to cDNA. First, the mRNA is converted to cDNA through reverse transcription. Initiation of reverse transcription may employ an oligo consisting of, from 3′ to 5′: any nucleotide (N), any nucleotide except thymine (A/G/C/), and a twelve- to eighteen-thymine oligo (anchored oligo dT) followed by a known sequence (primer P1). The use of anchored oligo-dT forces hybridization at the beginning of the polyadenylation sequence in the mRNA, removing the possibility of having more than eighteen adenosine nucleotides at the end of the PCR template, and reducing the size of the PCR template. See Thomas et al., 21 Nucleic Acids Res. 3915-16 (1993). The complementary strand of cDNA may be synthesized by the Gubler-Hoffman second strand synthesis method. Gubler & Hoffman, 25 Gene 263-69 (1983).

A restriction enzyme may then be used to cut the double-stranded cDNA at a known recognition site such that there is an overhang of at least four bases. An adapter primer with a complementary overhang attached to a double-stranded primer (primer P2), may then be ligated to the cDNA molecules. Primer P2 may contain a common 20 mer-22 mer primer, a random 6 mer-9 mer, and a 4-base overhang complementary to the restriction enzyme overhang. This uniquely tags each molecule with a random sequence, which will later be used to disambiguate cases where two beads were captured in a single microenvironment, causing both to have the same sequence amplified on them, and two beads that capture the different molecules of the same mRNA sequence and are amplified separately. The former will contain the same random 6 mer-9 mer sequence while the latter will not. There are three possible side ligation reactions: (a) the P2 adapter dimerization; (b) P2 ligates to the end of cDNA molecules that lack P1 sequence; and (c) cDNAs ligate to each other. The first two reactions result in molecules lacking P1, and the last reaction results in a molecule that lacks P2 primer. None of these three side reactions will produce a PCR template. The third side reaction may result, however, in lost potential sequencing template, and can be minimized by adding the P2 adapter in excess concentration. At this stage, all mRNA molecules are converted into double-stranded cDNA molecules with known sequence (P1 and P2) on each end. The present method may be distinguished from MPSS, which ligates a library of unique tags to the double-stranded cDNA molecules, such that each molecule has a unique sequence attached to it. Although this process allows for a standard PCR reaction from which homogeneous PCR products may be collected on a single bead with the appropriate tag, the embodiments provided for herein may prove more cost-effective.

The sequencing of the present invention may involve highly parallel 2-dimensional pyrosequencing conducted on a microscope slide (e.g., a glass imagable area) under a CCD or CMOS camera. When DNA polymerase incorporates a nucleotide into the complementary strand of the sequencing template, it releases a pyrophosphate (PPi) molecule. In pyrosequencing, this PPi is converted into ATP by ATP sulfurylase, producing energy required for luciferase to oxidize luciferin and generate light. In the past, the measurement of adenosine incorporation was problematic because ATP reacted with luciferase, creating a false signal. ATP was therefore substituted with deoxyadenosine alpha-thiotriphosphate (dATPαS), which is not recognized by luciferase but is incorporated efficiently by DNA polymerase, allowing the measurement of adenosine incorporation. Ronaghi et al., 1996. A further improvement added apyrase to degrade unincorporated nucleotides removed the need for a wash step during each cycle. Ronaghi et al., 281 Science 363-65 (1998). Further, the addition of a single-stranded DNA binding protein has simplified the optimization of the parameter in the protocol and allowed for longer sequence reads. Ronaghi, 2001.

One of the approaches of the present invention limits variability encountered in gene expression experiments and provides data sets that are comparable and reproducible across laboratories. For example, instead of relying on relative, semi-quantitative expression levels for each gene, the present invention provides for an actual count of the number of each type of mRNA molecule in the sample, greatly facilitating the search for biomarkers and combining data sets from different laboratories.

In another aspect, the present invention provides a platform that runs the various molecular biology reactions to completion in compartmentalized microenvironments, making the resultant data digital (a molecule is present or absent). This process removes variability introduced by individual reactions. A further aspect of the present invention identifies the sequence of each molecule in the sample, removing the need to have pre-existing sequence knowledge or collections of specific probes. Therefore, all genes have an equal chance of being counted, including novel ones, regardless of which laboratory is collecting the data.

In another aspect, the present invention creates a homogenous sequencing template population affixed to a single magnetic bead from each starting mRNA molecule. The sequencing template is sequenced in a high throughput manner to obtain a small amount of sequence (sequence tag) corresponding to each mRNA in the starting sample. The sequence tag is of sufficient length to uniquely map the tag to a gene in a known genome.

In another aspect, the present invention involves self-assembly of very small microbeads immobilized on a glass slide and imaged microscopically. This method allows for three-order of magnitude greater scale than previous bead-based technologies and thus application to capture of large global datasets, for example in gene expression profiling. The method involves the application of commodity equipment, leading to greatly reduced cost-per-sample.

In another aspect, the methods of the present invention may obtain sequences from as little starting material as a single cell, allowing cell-specific targeting of expressed sequence tag generation by, for example, laser-capture microdissection of histologically stained tissues or fluorescence activated cell sorting (FACS). This allows the selection and direct sequencing of sequences differentially expressed, allowing focus on the genes pertaining to a specific state.

In an aspect, the present approach works by isolating each mRNA molecule and amplifying its sequence to create a homogeneous population of sequencing templates bound to a bead. All of the beads may be sequenced simultaneously using pyrosequencing. Primers with known sequence must be present on both sides of each molecule to allow for PCR amplification. One step in this process requires cutting each cDNA molecule with a restriction enzyme. There are several criteria that may be considered in choosing an optimal restriction enzyme: (a) there must be a manufacturer of the enzyme; (b) the cut must result in an overhang (sticky end) with at least four bases; (c) there should be minimal degeneracy in the restriction enzyme recognition sequence; and (d) the number of sequences not cut should be minimized, as should sequences cut at less than twenty bases from the 3′ end and sequences cut over 2000 bases from the 3′ end. Criteria (b) and (c) ensure that the ligation of the adaptor primer is efficient. If the overhang sequence is less than four bases, it may be difficult to hybridize the adaptor primer to the double-stranded DNA. If the overhang is highly degenerate, the effective concentration of adaptor primers with the correct overhang is drastically reduced. Criteria (d) may be important to maximize the number of genes that can be detected in the present invention. Genes lacking the restriction enzyme recognition site will not have adaptor primer P2 ligated to the 5′ end. Genes that are cut less than twenty bases from the poly A 3′ tail may not have enough sequence to uniquely identify them. Genes that are cut at more than 2000 bases from the poly A 3′ tail may not have efficient PCR amplification.

All of the restriction sites in REBASE (Roberts & Macelis, 29(1) Nucleic Acids Res. 268-69 (2001)) meeting criteria (b) were used to digest all of the sequences in the Human RefSeq database in silico. Fruitt et al., NCBI Reference Sequence (RefSeq): a curated non-redundant sequence database of genomes, transcripts and proteins, 33 Nucleic Acids Res. D501-04 (2005). The RefSeq sequences were filtered such that no sequences have more than 150 identical bases in common. The distributions of the site distances from the 3′ end of each gene are shown in Table 1. Note that top twenty recognition sites that minimize unacceptable fragment sizes are shown. Excluding overhang sequences that do contain degenerate bases, there are two options: Mbol (GATC) and FatI (CATG) and their isochizomers. The other two sequences are the equivalent 3′ overhang sequences. MboI has fewer fragments of insufficient length what compared to FatI; the distribution of fragment sizes for FatI is smaller, however, producing more efficient PCR reactions. Following this example analysis, FatI presents a restriction enzyme that may be useful in the present invention. EcoRII and SsolI may also be useful.

TABLE 1
Fragment Size Distributions of Selected Restriction Enzymes.

| General Information | | | | Unusable Fragments | | | | Fragment Sizes | | |
|---|---|---|---|---|---|---|---|---|---|---|
| Name | Overhang | End | Deg.¹ | No cut | <20 | >2000 | Total | Median | Mean | Max |

| | | | | | | | | | | |
|---|---|---|---|---|---|---|---|---|---|---|
| SecI | CNNG | 5′ | Y | 190 | 472 | 208 | 5.1% | 222 | 377 | 5580 |
| EcoRII | CCWGG | 5′ | Y | 230 | 470 | 213 | 5.3% | 230 | 379 | 5101 |
| SsoII | CCNGG | 5′ | Y | 156 | 584 | 182 | 5.4% | 209 | 354 | 5101 |
| TspRI | NNGTSA | 3′ | Y | 363 | 579 | 27 | 5.7% | 231 | 323 | 2726 |
| | CNN | | | | | | | | | |
| Chai | CTAG | 3′ | N | 441 | 502 | 106 | 6.1% | 323 | 435 | 3708 |
| MboI | GATC | 5′ | N | 441 | 502 | 106 | 6.1% | 323 | 435 | 3708 |
| UnbI | GGNCC | 5′ | Y | 335 | 432 | 349 | 6.5% | 278 | 462 | 5333 |
| NlaIII | GTAC | 3′ | N | 161 | 1089 | 10 | 7.3% | 164 | 239 | 3254 |
| FatI | CATG | 5′ | N | 161 | 1089 | 10 | 7.3% | 164 | 239 | 3254 |
| MaeIII | GTNAC | 5′ | Y | 552 | 744 | 64 | 7.9% | 251 | 361 | 3156 |
| SduI | HCGD | 3′ | Y | 657 | 307 | 443 | 8.2% | 333 | 519 | 6729 |
| Tsp451 | GTSAC | 5′ | Y | 1164 | 409 | 391 | 11.5% | 406 | 566 | 5114 |
| SfeI | TRYA | 5′ | Y | 1410 | 207 | 544 | 12.6% | 494 | 655 | 7381 |
| Vpak11AI | GGWCC | 5′ | Y | 1109 | 209 | 948 | 13.2% | 476 | 699 | 6408 |
| BbvI | NNNN | 5′ | Y | 1133 | 208 | 961 | 13.4% | 524 | 738 | 5747 |
| StyI | CWWG | 5′ | Y | 1487 | 173 | 833 | 14.5% | 510 | 706 | 7145 |
| FokI | NNNN | 5′ | Y | 1916 | 146 | 962 | 17.6% | 585 | 774 | 9095 |
| StsI | NNNN | 5′ | Y | 1916 | 146 | 962 | 17.6% | 585 | 774 | 9095 |
| Sthl321 | NNNN | 5′ | Y | 1466 | 271 | 1624 | 19.6% | 537 | 844 | 9420 |
| TaiI | TGCA | 3′ | N | 2084 | 342 | 1045 | 20.2% | 584 | 791 | 8470 |

Deg¹ = degenerate sequence

A feature of the present invention recovers a sequence from each molecule of the starting mRNA samples. This sequence may uniquely identify a single gene. An efficient approach minimizes the number of sequencing cycles performed (to minimize cost), while obtaining enough bases to map each sequence to a unique gene. One approach to determining how many bases must be sequenced involves comparing the ideal results of sequencing all genes in silico starting from the 3′ most restriction enzyme recognition site for various lengths to the number of unique gene hits returned. Human RefSeq, a curated database of all known genes, was made non-redundant by keeping only one sequence of r each set of sequences with more than 150 bases identical. The non-redundant set was digested in silico by FatI (the sequences were cut at the enzyme recognition sequence CATG) and only the 3′ most sequences were kept. Each sequence was truncated to a given length (x-axis on FIG. 5) and compare back to non-redundant Human RefSeq. The percent of all sequences that returned a unique hit in RefSeq was computed (y-axis in FIG. 5). FIG. 5 shows that seventeen bases of sequence may be necessary to uniquely identify 99% of the genes. With pyrosequencing, the present approach can sequence up to fifty base pairs potentially allowing for uniquely identifying 99.8% of all genes. Thus, twenty bases is a reasonable objective, within the limits of pyrosequencing, and above the threshold for uniquely identifying over 95% of all genes.

The following examples illustrate various methods for compositions in the treatment method of the invention. The examples are intended to illustrate, but in no way limit, the scope of the invention.

### EXAMPLES

### Example 1

### Conversion of RNA to DNA

A cDNA molecule is created from each mRNA with a specific primer (P1) attached to the 5′ end via reverse transcription (Superscript III) (Invitrogen, Carlsbad, Calif.) using oligo-dT with primer P1 attached to the 5′ end. Each cDNA is converted into a double stranded DNA (dsDNA) using the Gubler-Hoffman Second Strand Synthesis method (DNA Ligase, RNAse H, T4 Polymerase, DNA Polymerase I) (Invitrogen). Each cDNA is cut with a restriction enzyme so that there is a 5′ overhang with known sequence, e.g., FatI, which leaves a 5′ overhang of CATG. Each dsDNA is ligated with a complementary oligo with a 5′ overhang of CATG connected to a primer P2 (see FIG. 1). Primer P2 is not phosphorylated, so the resultant product can not concatemerize. Adding adapter P2 in excess concentration minimizes cDNA chimera formation. This yields dsDNA molecules that are flanked with primers P1 and P2 on the 5′ ends.

### Example 2

### Bead Synthesis and Emulsion PCR

The PCR product must be attached to some solid substrate to maintain physical locality when the sequencing template is removed from the emulsion. One approach provides beads coated with the 3′ primer sequence P1 (see FIG. 2) such that the PCR product is bound to the bead. After the PCR reaction, the beads can be isolated and prepared for sequencing.

Briefly, beads coated with bound primer P1 are synthesized by mixing super magnetic beads coated with covalently bound streptavidin (Dynabeads M-280 or MyOne C1 or M450 tosyl activated with subsequent streptavidin incorporation) with P1 oligos modified on the 5′ end with dual biotin groups separated by a six-carbon linker with a spacer arm (to reduce steric hindrance and allow the oligonucleotides to be cleaved). After binding has occurred, the beads are washed thoroughly to remove unbound oligonucleotides. Oligonucleotides with a single biotin group may dissociate from the beads when the temperature is cycled in the PCR reaction, whereas oligonucleotides with the dual biotin group are more stable under PCR cycling. Dressman et al., 100 P.N.A.S. 8817-22 (2003).

A water-in-oil emulsion is created as described (Dressman, 2003; Tawfik & Griffiths, 1998) in which the oil phase consists of 4.5% Span 80, 0.40% Tween 80, and 0.05% Triton X-100 in mineral oil. The aqueous phase consists of 67 mM Tris-HCl (pH8.8), 16.6 mM NH₄SO₄, 6.7 mM MgCl₂, 10 mM 2-mercaptoethanol, 1 mM dATP, 1 mM dCTP, 1 mM dGTP, 1 mM dTTP, 0.05 μM forward primer P1, 25 μM reverse primer P2, 45 U of Platinum Taq (heat activated), template DNA and oligonucleotide-coupled beads. A small amount of unbound primer P1 is added in the aqueous phase to help initiate the PCR reaction by providing more unbound template. The microemulsions are created by the addition of aqueous phase solution to the oil phase one drop at a time. This addition is performed in a one minute period under constant stirring at 1400 rpm. After the addition of the aqueous phase, the mixture is stirred for 30 min. The PCR reaction does not start until after the emulsion has been formed because it is heat activated.

The aqueous phase contains beads and template DNA in a concentration such that 1:100 microdroplets contain a single DNA template molecule and 1:100 microdroplets contain a single bead. At these concentrations, only 1 in 100 microdroplets with a DNA will contain a bead. Therefore, only 1/100th of the starting template molecules are sequenced. The probability of a microdroplet with a bead and a DNA molecule containing yet another molecule is about 1/200 (derived from the binomial distribution). Therefore, only 0.5% of the sequenced data is unusable (because a bead coated with more two different DNAs yields a sequence not specific to any one gene). Other embodiments of the invention may use different numbers of beads, compartments, and molecules to achieve the desired molecule sampling rates and unusable bead tolerances. These ratios of beads to compartments and molecules to compartments here are given as example. Other ratios are described above.

The emulsion is aliquoted into PCR tubes and polymerase chain reaction is run in each microdroplet with primer P2 in solution and primer P1 bound to the bead. This process results in beads coated with dsDNA with the template: 5′-P1-polydT-cDNA_sequence-RE-antisense_P2 where RE is the restriction enzyme site (e.g., CATG for FatI restriction enzyme) if the sequencable DNA was produced from RNA.

The double stranded DNA on the beads is denatured and washed, leaving only the single stranded cDNA bound to the bead with biotin. The beads are then ready for sequencing. This entire process, including sequencing, may take about fifteen hours.

### Example 3

### Bead Immobilization

As noted previously, it is desirable to immobilize the beads to the imagable surface such that they do not move when reagents flow over them. If the beads move, it will be impossible to register the sequential images and determine the sequence of nucleic acid on each bead. Beads can be immobilized to the slide in a number of ways. One method of immobilizing beads is streptavidin-biotin binding of the beads to a biotinylated protein and covalent binding of carboxyl groups and amine groups of the protein to glass via silation of the glass with a reactive group containing silane (such as 3-Aminopropyltriethoxysilane (APTES)). A second method is silanization of glass with APTES but modification of the 3′ end of the DNA on the bead by ligation with a nucleotide containing a 3′ primary amine group and covalent bonding to the slide through amine-ester bonds.

These procedures provide a dense monolayer coating of beads. Also, the non-uniform layout of the beads provides fiduciary marks for image.

### Example 4

### Flow Cell Production

A flow cell is created using a soft lithography process with polydimethylsilicone (PDMS). A black and white mask is created such that white features define the flow cell. A silicon substrate (e.g. a silicon wafer) is coated with a 50 micron layer of photo resist and UV light is applied through the mask onto the photo resist. The wafer is washed and cleaned leaving photo resist only where the UV light penetrated the mask and polymerized the photo resist. The process produces the master template. The master is silanized to prevent PDMS from polymerizing to the mask. PDMS is poured over the master and allowed to polymerize for 4 hours at 65° C. Once cured, the PDMS is removed from the master and silanized. A new layer of PDMS is poured over the first PDMS mold and allowed to polymerize for 4 hours at 65° C. This produces the flow cell. If multiple flow cells are laid out on the master, then the flow cells are isolated from each other using a razor blade.

### Example 5

### Flow Cell Operation

Holes are punched though the inlet and outlet ports with a 23 gauge needle and tubing is attached to the ports. The needles are blunted and sharpened from the inside with a carbide tip. The punch cores are removed from the needle with #11 wire. For example, PEEK tubing ( 1/32″ OD, 350 um ID) is used because it is stiff and has a small inner diameter to minimize dead volume in the flow cell apparatus.

### Example 6

### Reagent Delivery

Reagents are stored in syringes contained in syringe pumps. The syringe pumps are daisy-chained together in a network and the first pump is attached to the computer. All reagent syringe pumps are connected to computer-controlled valves. One end of the valve (called the flow cell inlet) is fitted with 30 gauge needles and PE-10 tubing. The tubing is connected to a type of manifold valve called a PerfusionPencil™ manifold (AutoMate Scientific., Inc., Berkeley, Calif.). The manifold joins all of the streams of reagents into a single stream through a needle (350 umOD, 250 um ID), which is connected to the flow cell inlet by tubing. The other end of the valve (called the reagent container inlet) is connected to a container with excess amount of reagent. The syringe pumps can refill by switching the valve to the reagent container inlet and withdrawing solution. When reagent is pumped into the flow cell, the valve is turned to the flow cell inlet position to provide reagent to the PerfusionPencil™ manifold. A syringe is attached to the flow cell outlet by tubing. This syringe applies a constant negative pressure by withdrawing from the flow cell outlet as reagents are pumped into the flow cell from the inlet. This syringe has a valve which can switch between a waste container and the flow cell outlet. When the syringe is full, it removes the waste by switching to the waster container inlet position and infuses its contents into the container. The computer uses recommended standard 232 (RS-232) communication to control all syringes and valves.

### Example 7

### Sequencing Apparatus

FIG. 1 shows the layout of a sequencing apparatus of an embodiment of the present invention: six pumps with six valves pump washing fluid, mineral oil, and the four nucleotides. The tubes from the pump to the bead container have mechanical valves that stay closed unless pressure builds behind them. Beads are immobilized in a monolayer in the imagable area as described previously. The monolayer is created at proper concentrations (e.g., 9×10⁵ beads/μL). The reagents are delivered to the bead container by the bead pump. Nucleotide master mixes (including the pyrosequencing reagents) are added to the solution one at a time in the repeating sequence: CTP master mix; ATP master mix; TTP master mix; GTP master mix. Reagents may be introduced and evacuated through the Waste Out Flow line, which may be fashioned into a multipurpose line to push and pull reagents. Alternatively, another separate line may be added for mixing reagents.

A Canon EOS 40D SLR Camera with a prime 1-5× Macro lens is used to image the flow cell from the bottom. With the f/2.8, which is equivalent to the numerical aperture of 0.18, the diffractive-limited resolution of the lens is 3.8 μm, which is sufficient to resolve 4.5-μm beads described in this invention. The image will be integrated for 0.1 s and stored on a computer.

Sensitivity and Resolution: 4.5 μm beads are covered with 10 million molecules of DNA. The 4.5 μm beads are magnified 3.2×(prime macro lens supports 1-5× zoom) and imaged with a 5.7 μm pixel CMOS chip so each bead covers 2.5×2.5 pixels. The camera is 10.1 megapixels so at maximum density 1.6 million reads are obtained. In 0.1 s, 80,000 photons is emitted from a bead in a 15 μm radius. An optical system with an f/stop of 2.8 may capture 0.8% or 640 photons. The light is spread over a circle of radius 8.4 pixels (15 um*3.2×/5.7 um per pixel) or 223 pixels. This produces an average of 2.9 photons per pixel in this region. At a quantum efficiency of 90% and internal gain of 5×, this yields 13 electrons/pixel, which is 3.0× the read noise floor (4.3e⁻ rms). Anti-reflection coating of the optical elements provides for high light transmission through the glass in excess of 90%; therefore, losses due to Fresnel reflection can be safely disregarded in these rough estimates. Also, f/# of 2.8 corresponds to numerical aperture NA of ˜0.18, which provides resolving power of 1.22 λ/NA=3.8 μm at λ=560 nm, which is sufficient to discern individual 4.5 μm-wide beads.

### Example 8

### Localization of Signal Source in Image

There are two ways to localize the signal to the correct bead given the image of the signal dispersed by diffusion of pyrophosphate. The first method, binary thresholding, exploits the fact that the highest concentration of signal is at the bead surface (because as the pyrophosphate and ATP diffuse, their concentration decreases). In this method, a threshold is chosen above the overall noise level in a part of the image. All pixels above the threshold are considered “on” and the others are “off”. The resulting mask can be applied to a brightfield image of the beads to localize the signal to the beads. This method can cause false negatives if the signal is too disperse; and false positives around beads that contain homopolymer sequence matching the nucleotide currently entering the system. Taking multiple pictures in succession and integrating them to find an image that does not have too much dispersion can alleviate the former problem. The latter problem can be addressed by using 3′-prime capped oligos.

A second method is to use image deconvolution. Essentially, the observed image is the convolution of the actual point source image (i.e. the true image that is desired) and a point source function that blurs the image. This is equivalent to pixel by pixel multiplication of the point source function and the image in the Fourier domain. Thus deconvolution is the process of dividing the captured image by the point source function in the Fourier domain and subsequently taking the inverse Fourier transform. The point source function can be calculated by imaging a single bead at different time points. With a known “correct” image, the point source function can be derived from the captured image. Deconvolution in the absence of noise provides a perfect recreation of the original image. However, there is noise inherent in the imaging system (mostly due to the camera A/D conversion). As a result, only the spatial frequencies of the desired image whose values exceed the noise floor of the imaging system can be recovered. The amount of information recovered is a function of the signal to noise ratio. The system has been engineered such that the signal to noise ratio of the majority of the imaged data is above 2.

### Example 9

### Optics

A large area CCD or CMOS sensor can be used to capture the light signal by integrating photons over the course of the reaction. There can be multiple flow cells used, but only one flow cell will be imaged at a time. The optics magnification can be such that each bead will be represented by 2.5 pixels (to satisfy the Nyquist limit of 2+ε pixels/bead) and with an NA of >0.15 or f#<1/(2*NA)=3.3 to satisfy Albe's equation for image resolvability (d=4.5 um<1.22*0.560 um/NA===>NA>0.15). In one example, a 5.71 μmpixel, 10.1 megapixel Canon camera is used with a 3.2× magnification lens (3.2 mag*4.5 μm bead=14.4 cm image=2.5 pixels/bead). The lens in this example is implemented as a 1-5× prime macro lens on an SLR camera.

### Example 10

### Sequencing Cost and Throughput Analysis

Throughput: It is anticipated that each flow cell will hold about 1.6 million beads. The system is to be imaged every 0.1 s. It is expected that 0.05 s will be allowed for reagent delivery of 0.5 μL (this is well within the bounds of the syringe pumps). It is estimated that the total time for delivery, reaction, and washing is 6 s per base (this is an overestimation). Thus, four cycles (A, T, G, C) should take 24 s, including washes. On average this yields 2.5 bases per cycle yielding 600 megabases per hour (1600000 beads*2.5 bases/bead/24 seconds*3600 seconds/hour). Multiple flow cells are expected to further increase throughput by running the wash steps in parallel.

Cost: At current prices, pyrosequencing reactions cost about $330 per 33 mL of reagents (after enzyme/substrate reconstitution and 10× dilution with annealing buffer) or $0.01 μl. The total volume of the flow cell including inlet path is expected to be about 0.5 μl. Four cycles should use 2 μL and yield 2.5 bases for 1.6 million beads or 4 megabases. Therefore, the pyrosequencing reactions are currently calculated to cost $0.008/megabase. There are also cycle independent costs of beads (approximately $0.03/million beads) and emulsion PCR reagents (approximately $0.82/r×n). It is estimated that 100 cycles should produce approximately 250 million bases of sequence costing $0.008*250=$2.00 for sequencing, $0.48 for beads (using a 10:1 ratio of molecules per emulsion compartment, 16 million beads are necessary to yield 1.6 million beads with template), and $0.82 for the emulsion PCR reaction. This yields $3.30/250 million bases or $0.01/megabase. Additional flow cells only affect throughput and should not change the cost.

### Example 11

### Identifying Unusable Beads (Beads with Heterogeneous mRNA Populations)

In an aspect of the invention, it is important to flag beads with non-homologous sequences, which will yield incorrect tag information, so that their data is not used in downstream analysis. Additionally, it is relevant to quality control purposes to count how many beads have unusable sequences. If there are two or more sequences on a single bead, the bead will incorporate bases at a much more rapid rate than normal. An in silico simulation of twenty cycles of pyrosequencing (C, A, T, G in one cycle) was carried out. The distribution of the number of nucleotide washes between the next incorporation of a base into the sequence of the bead was much different between homologous and non-homologous solutions (see FIG. 3). This simulation ran twenty cycles of pyrosequencing (i.e. 80 nucleotide washes) and was rerun one million times. For a homogeneous population, the means number of times for zero, one, two, or three washes before incorporation is 13 and the mean of the total number of bases read is 52 bases. For non-homogenous populations (e.g., two different sequences on a bead) the average total number of bases read is 104 bases. In the very rare instances where the distributions of the total number of bases read cannot distinguish a non-homogeneous population from a homogenous populations (about 0.01%), the distributions of times one and three washes occur before next incorporation are very different and can distinguish between the homogeneous and non-homogeneous in all cases. For non-homologous populations, there are very few times that three washes occur before a base is incorporated into a sequence (on average, only three times per simulation). Additionally, for non-homologous populations, there are significantly more times where base incorporation was separated by only one nucleotide wash (average of 42 times per simulation). These distributions can distinguish between beads with homogeneous and non-homogeneous populations.

### Example 12

### Image Registration

Initially, all beads will emit light when the first four known bases are tested (CATG, from the restriction enzyme site). This provides the initial position of each bead. Registration marks are made on the sides of the slide creating a Cartesian coordinate system. The beads are positioned and tracked in software with distances from the registration marks. The beads should not move significantly because they are immobilized. However, small jitter (<1 μm per scan) will be accounted for in software. Brightfield images take before the reaction begins are used to align the images taken during the reaction and the brightfield images can be registered with each other using a simple correlation filter. Additionally, all of the image information can be saved to the hard disk. After the experiment is complete, if the image registration is off, the resulting sequence will not match known genomic sequence. The registration process can be redone such that the resulting sequence more closely matches known sequence. Alternatively, the beads with sequencing template can be packed along with empty beads. This way beads with sequencing template can be tracked even in the face of significant jitter.

Numerous modifications and variations of the present invention are possible in light of the above teachings. It is therefore to be understood that within the scope of the appended claims, the invention may be practiced otherwise than as specifically described herein.

1. A method for sequencing a nucleic acid molecule, comprising:
(a) providing a sequencer, comprising:
(i) a reservoir; and,
(ii) a microfluidic flow cell, comprising: a flow chamber, comprising:
a planar imagable area; and,
a plurality of beads immobilized onto the planar imagable area;
wherein:
the plurality of reservoirs is fluidly connected to the flow cell; and,
a substantial portion of the beads, further comprise: a plurality of nucleic acid primers attached thereto, wherein the nucleic acid primers present on an individual bead are homogeneous;
(b) contacting the target nucleic acid molecule with said beads and pyrophosphate sequencing reagents, comprising:
a nucleotide triphosphate;
a polymerase;
a pyrophosphate to ATP converting enzyme; and,
an ATP detecting enzyme; and
(c) detecting the resulting optical signals, wherein each optical signal is indicative of a reaction of pyrophosphate sequencing reagents with a target nucleic acid molecule on a bead, thereby sequencing the nucleic acid.

2. The method of claim 1, wherein the microfluidic flow cell further comprises:
a first fluid inlet fluidly connected to the flow chamber and fluidly connected to the plurality of reservoirs; and,
a first fluid outlet fluidly connected to the flow chamber.

3. The method of claim 1, wherein the first fluid inlet and first fluid outlet are connected to the same surface of the flow cell and are separated by the imagable area.

4. The method of claim 1, wherein the planar imagable area comprises glass.

5. The method of claim 1, wherein the space between the imagable area and the wall of the flow cell is from 5 μm-100 μm.

6. The method of claim 1, wherein the contacting is performed by delivering the pyrophosphate sequencing reagents from the plurality of reservoirs to the flow chamber whereby the nucleic acids are exposed to the reagents.

7. The method of claim 6, wherein the contacting further comprises sequential delivery of homogeneous nucleotide triphosphates.

8. The method of claim 1, wherein the pyrophosphate sequencing byproduct is detected by contacting it with an ATP sulfurylase under conditions that allow for formation of ATP.

9. The method of claim 8, wherein the ATP sulfurylase is a thermostable ATP sulfurylase.

10. The method of claim 1, wherein the pyrophosphate sequencing byproduct is detected by contacting it with a pyruvate orthophosphate dikinase under conditions that allow for formation of ATP.

11. The method of claim 10, wherein the pyruvate is a thermostable pyruvate orthophosphate dikinase.

12. The method of claim 1, further comprising washing the flow cell with a wash buffer between each delivery of a nucleotide triphosphate.

13. The method of claim 1, wherein the nucleic acid molecules are attached to the beads via their 5′ ends via a biotin-streptavidin binding linkage.

14. The method of claim 1, wherein the beads are immobilized onto the imagable surface via a binding pair or a chemical bond.

15. The method of claim 4, wherein the beads are immobilized to the imagable glass surface via a strepavadin-biotin-protein-silanyl linkage between nucleic acid bound to the bead and the imagable glass.

16. The method of claim 4, wherein the beads are immobilized to the imagable glass surface via a 3′ nucleic acid comprising a primary amine group-silanyl linkage.

17. The method of claim 1, wherein the diameter of the beads is from 1 μm-20 μm.

18. The method of claim 1, wherein the beads are packed such that the free space is between the beads is less than 20% of the total imagable area.

19. The method of claim 1, wherein the ATP detecting enzyme is luciferase, which produces light for detection.

20. The method of claim 21, wherein the luciferase is a thermostable firefly luciferase.

21. The method of claim 1, wherein the signal detection is performed by a CMOS camera.

22. The method of claim 1, wherein the optical signals from the pyrophosphate sequencing reaction are imaged before the reagents and byproducts diffuse far enough away from the bead incorporating the nucleotide sequence that the light can no longer be localized to that specific bead.

23. The method of claim 1, wherein the reaction is imaged within 10 Ms-1000 Ms.

24. The method of claim 1, wherein the optical signal is light and signal deconvolution is used to localize the light signal to a bead.

25. The method of claim 1, wherein the sequencer comprises a plurality of flow cells.

26. A sequencer for sequencing a target nucleic acid molecule, comprising:
(i) a reservoir; and,
(ii) a microfluidic flow cell, comprising: a flow chamber comprising:
a planar imagable area; and,
a plurality of beads immobilized onto the planar imagable area;
wherein:
the plurality of reservoirs is fluidly connected to the flow cell; and,
a substantial portion of the beads further comprise: a plurality of nucleic acid molecules attached thereto, wherein the nucleic acid molecules present on an individual bead are homogeneous;

27. A kit for sequencing a nucleic acid molecule, comprising:
a. a polymerase;
b. a pyrophosphate to ATP converting enzyme;
c. an ATP detecting enzyme;
d. nucleotides, or optionally nucleotide analogues, optionally including, in place of dATP, a dATP analogue which is capable of acting as a substrate for a polymerase but incapable of acting as a substrate for a said pyrophosphate to ATP converting enzyme;
e. optionally dideoxynucleotides, or optionally dideoxynucleotide analogues, optionally ddATP being replaced by a ddATP analogue which is capable of acting as a substrate for a polymerase but incapable of acting as a substrate for a said PPi-detection enzyme;
f. optionally deoxynucleotides or dideoxynucleotides capped on the 3′ side with a 2-nitrobenzyl moiety to prevent successive incorporation of nucleotide in homopolymeric regions of DNA sequence.
